(19) United States
(12) Patent Application Publication
(10) Pub. No.: US 2012/0172696 A1
(43) Pub. Date: Jul. 5, 2012

(54) Flexible Conductor Carrier for Catheter and Catheter Fitted with a Conductor Carrier

(75) Inventors: Bengt Källbäck, Taby (SE); Malin Thomsen, Hagersten (SE)

(73) Assignee: CATHPRINT AB, Stockholm (SE)

(21) Appl. No.: 13/395,068

(22) PCT Filed: Jul. 1, 2010
(86) PCT No.: PCT/SE2010/000187

§ 371 Date: Mar. 8, 2012

(30) Foreign Application Priority Data
Sep. 10, 2009 (SE) 0901166-9

(51) Int. Cl. A61B 5/04

(52) U.S. Cl. 600/373

(57) ABSTRACT

The invention relates to a flexible conductor carrier (5, 5a-b, 6, T) comprising an elongated inserted section (5, 5a-b, 6) intended to be arranged in a lumen (2a-b) in a flexible catheter. The inserted section (5, 5a-b, 6) comprises a flat section (5, 5a-b) and a rolled section (6). The flat section provides higher tolerance to bending than a fully-rolled counterpart. Typically, at least one electrode (4a-b) is fixed around the rolled section (6).

The present invention relates to a flexible conductor carrier and a catheter equipped with such a conductor carrier according to the preamble of the claims.

### BACKGROUND OF THE INVENTION

Flexible catheters with conductors running along the inside of the catheters are known. Such a flexible catheter is described in WO2007139479. The document describes an elongated, rectangular flexible circuit board that is rolled to form a cylindrical element and constitutes the catheter. The cylindrical conductor is flexible but the electrical conductors on the printed circuit board may be broken or damaged if bent too sharply. In clinical situations, it is often necessary to bend a catheter, which can cause the conductor leads on the cylindrical multi-conductor to break.

One purpose of the invention is therefore to provide a flexible conductor carrier, which tolerates a greater degree of bending than prior art for such conductor carriers, and a catheter equipped with such a conductor carrier.

Another purpose of the invention is to provide a flexible conductor carrier, which has a greater degree of flexibility than prior art for such conductor carriers, and a catheter equipped with such a conductor carrier.

These and other purposes are achieved through a flexible conductor carrier for a catheter and a catheter equipped with such a conductor carrier in accordance with the characteristic features of the independent claims.

### SUMMARY OF THE INVENTION

The invention relates to a flexible conductor carrier 5, 5a-b, 6, 7, at least one elongated inserted section 5, 5a-b, 6 intended to be arranged in a lumen 2a-b in a flexible catheter. The flexible conductor carrier is typically made of a flexible printed circuit board. The inserted section 5, 5a-b, 6 includes at least one flat section 5, 5a-b and one rolled section 6. The flat section offers the benefit of a higher tolerance to bending than its rolled counterpart. There typically exists at least one electrode 4 a-b around the rolled section 6.

The flat section 5, 5a-b can in a particularly advantageous embodiment comprise two elongated sections that mostly extend parallel to each other, providing space for additional conductors and hence, for additional electrodes.

The invention relates to such a conductor carrier 5, 5a-b, 6, 7 where the inserted section 5, 5a-b, 6 includes several flat sections 5, 5a-b and rolled sections 6 in sequence, so that electrodes can be placed at a number of positions along the catheter with flexible, flat sections of conductor carriers between them.

The invention relates to a catheter equipped with such a conductor carrier 5, 5a-b, 6, 7.

### BRIEF DESCRIPTION OF THE DRAWINGS

FIG. 1 shows a curved catheter according to prior art.

FIG. 2 shows a first embodiment of a cylindrical conductor carrier in the rolled state.

FIG. 3 shows the first embodiment of the conductor carrier in the flat state.

FIG. 4 shows a second embodiment of the conductor carrier in the flat state

FIG. 5 shows a catheter with the first embodiment of the conductor carrier inserted in a lumen.

### DESCRIPTION OF PREFERRED EMBODIMENTS

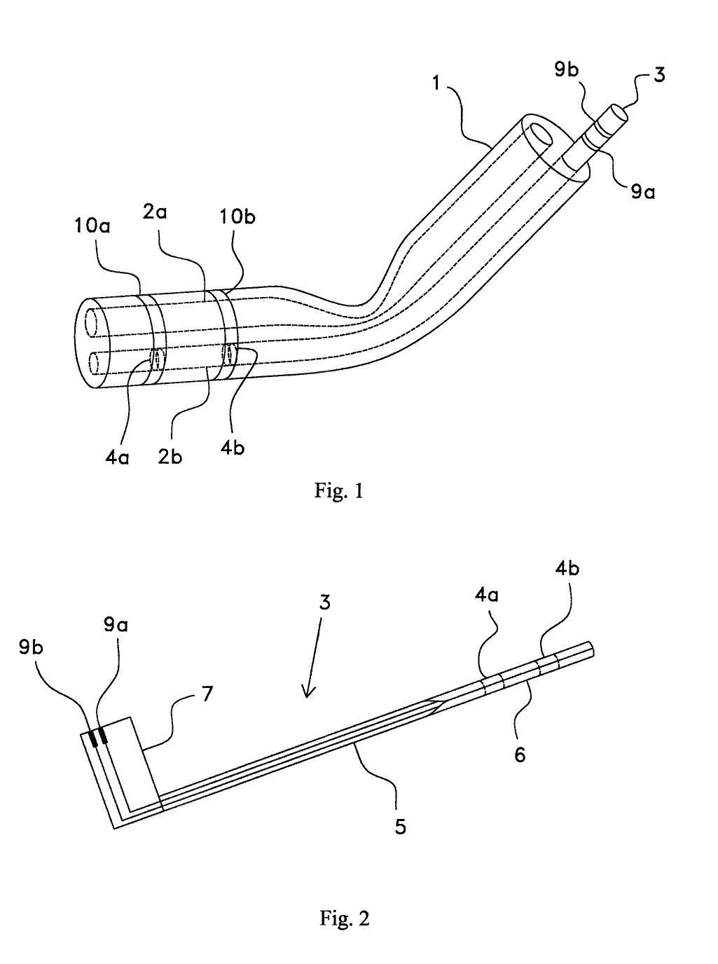

FIG. 1 shows a catheter with a curved conductor carrier designed according to known techniques. The catheter is made of a cylindrical rod 1 of a flexible material, with two cylindrical lumens 2a-b, that extend along the length of the catheter. The catheter is designed to transfer fluid through the lumens or alternatively to contain a conductor carrier for the electrodes in the lumens. Arranged in one lumen 2b is a conductor carrier 3 around which two internal electrodes 4a-b are fixed. The internal electrodes are electrically connected to external electrodes 10a-b, for measurement purposes or for applying energy to an organ via the conductor lines extending down the catheter to electrodes 9a-b.

Conductor carriers according to prior art consists of flat, rectangular pieces of flexible printed circuit rolled into a cylindrical shape and is fixed in this form by filling the cylinder with a flexible adhesive. Conductor carriers are very flexible when flat, but less flexible when rolled and copper conductors can be ripped off when bend sharply. During normal use, catheters are only bent moderately, which works well. However, a catheter can inadvertently be bent significantly, in which case the conductors on a conductor carrier of prior art can be damaged. In addition, catheters are frequently and deliberately bent sharply in order to cut off transfer of liquid or gas through the lumens, as illustrated in the figure. This can damage conductor carriers of prior art and this is one of the problems the conductor carrier of this invention solves. Another advantage is that the resultant catheter becomes more sufficiently flexible for use as a feeding tube, which is not always the case if the all parts of the internal catheter are rolled.

The risk of damage to the rolled section of the conductor carrier by excessive bending is eliminated by the conductor carrier being rolled only at sections where electrodes exist, with the rest of the conductor carrier is left flat. FIG. 2 illustrates a first embodiment of a cylindrical conductor carrier 3 according to the invention in the rolled state. The conductor carrier comprises a flat, unrolled section 5 and a rolled cylindrical section 6, both of which are arranged inside the catheter. A flat end of the conductor carrier, a broader section 7 is arranged outside the catheter; in this section the conductors are arranged to allow easy connection to electronic equipment. The other end of the flat section is connected to the rolled section 6, around whose external surface two internal electrodes 4a-b are arranged; these internal electrodes will be connected to external electrodes fixed to the outer surface of the catheter.

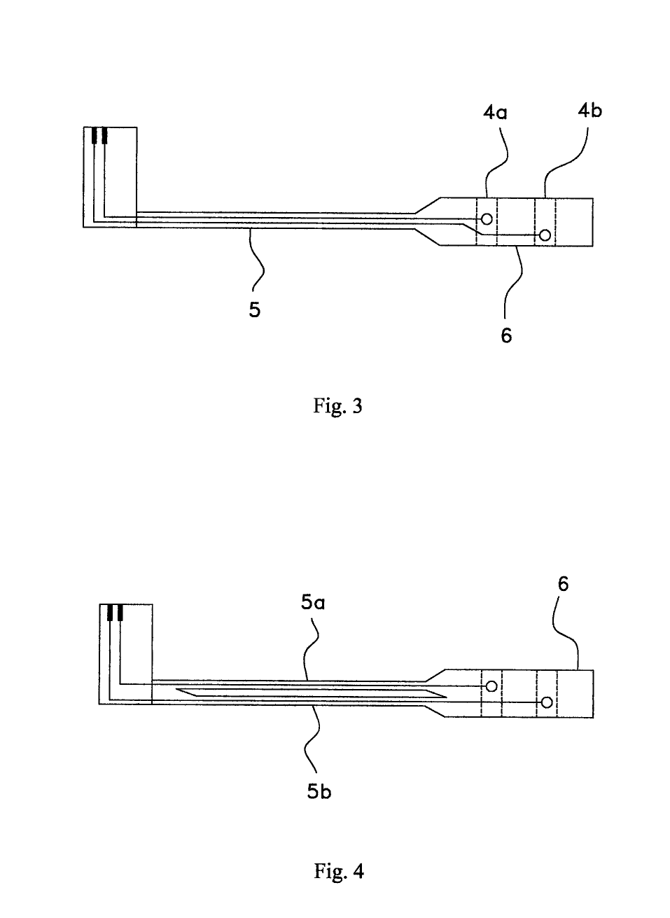

FIG. 3 shows the first embodiment of the conductor carrier in the flat state and clearly indicates the two sections 5 and 6, of which 5 remains flat and 6 is rolled after the rolling process. The section that becomes rolled 6 is wider than the flat section 5, preferably by more than three times, so that the diameter of the cylinder formed by the rolled section is equal to the width of the flat section. To reduce the risk of breakage in the transition between the two sections, the flexible circuit board is cut obliquely and not in rectangular shapes.

FIG. 4 shows the second embodiment of the conductor carrier in the flat state. Here, the flat section is divided into two parts 5a-b, which run parallel to each other along the length of the conductor carrier. When the broader section 6 of the conductor carrier is rolled, the one of the flat sections 5a-b is placed on top of the other flat section; as a result, the width of the flat section is narrower than its counterpart in the first embodiment. At the same time, a good level of flexibility is achieved and the resulting elasticity is comparable to that of the first embodiment. Just as in the first embodiment, the flat section 5a-b and the rolled section are intended to be inserted into a lumen in the catheter, while a wider section 7 connected to the flat section is intended to be arranged at one end of the catheter without being inserted into the catheter.

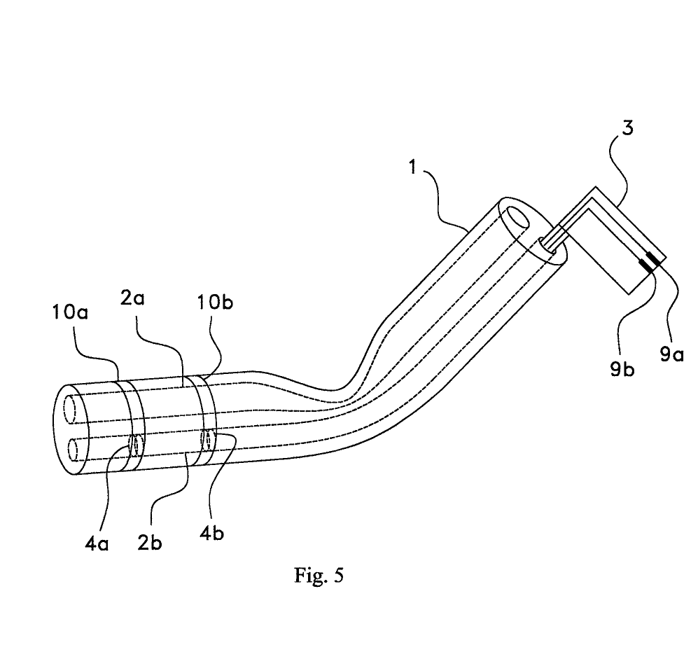

FIG. 5 shows the first embodiment of the conductor carrier inserted in lumen 2b of a catheter. The conductor carrier includes a flat section that is wider than the rest and on which the connection sections 9a-b are arranged. These connection sections 9a-b are in electrical connection with the two internal electrodes 4a-b.

The illustrated embodiments only show examples in which the conductor carrier includes one flat section and one rolled section that are inserted in the catheter. However, the conductor carrier may include a number of flat and rolled sections, depending on the length of the conductor carrier.

1. A flexible conductor carrier (5, 5a-b, 6, 7) including at least one elongated inserted section (5, 5a-b, 6) intended to be arranged inside a lumen (2a-b) in a flexible catheter (1), wherein the at least one insertion section (5, 5a-b, 6) comprises at least one flat section (5, 5a-b) and one rolled section (6).

2. A flexible conductor carrier (5, 5a-b, 6, 7) according to claim 1, wherein the at least flat section (5, 5a-b) comprises two elongated sections arranged mostly parallel to one another.

3. A flexible conductor carrier (5, 5a-b, 6, 7) according to claim 1, wherein the at least one electrode (4a-b) is fixed around the rolled section (6).

4. A flexible conductor carrier (5, 5a-b, 6, 7) according to claim 3, wherein the at least one conductor is connected to an electrode (4a-b) which extends from this electrode, along and to the end of the conductor carrier.

5. A flexible conductor carrier (5, 5a-b, 6, 7) according to claim 1, wherein the inserted section (5, 5a-b, 6) comprises multiple flat (5, 5a-b) and rolled (6) sections.

6. A flexible conductor catheter (1) containing at least one lumen (2a-b) that extends along the length of the catheter and is designed to contain a flexible conductor carrier (5, 5a-b, 6) including at least one elongated inserted section (5, 5a-b, 6) wherein the elongated inserted section comprises at least one flat section (5, 5a-b) and one rolled section (6).

7. A flexible conductor carrier (5, 5a-b, 6, 7) according to claim 2, wherein the at least one electrode (4a-b) is fixed around the rolled section (6).

8. A flexible conductor carrier (5, 5a-b, 6, 7) according to claim 7, wherein the at least one conductor is connected to an electrode (4a-b) which extends from this electrode, along and to the end of the conductor carrier.
